(19) United States
(12) Patent Application Publication
(10) Pub. No.: US 2021/0337800 A1
(43) Pub. Date: Nov. 4, 2021

(54) METHOD AND SYSTEM OF IMPROVING THE PERSONAL PROTECTIVE EQUIPMENT OF HEALTHCARE WORKERS AND THE GENERAL PUBLIC USING ANTIMICROBIAL TECHNOLOGY ADDITIVES

(71) Applicant: Sockdocs Inc., Great Neck, NY (US)

(72) Inventors: David Elliot SHAFFER, Great Neck, NY (US); Carlos ROSA, Sparta, NJ (US)

(73) Assignee: Sockdocs Inc., Great Neck, NY (US)

(21) Appl. No.: 17/232,350

(22) Filed: Apr. 16, 2021

Related U.S. Application Data
(60) Provisional application No. 63/018,827, filed on May 1, 2020.

(51) Int. Cl. A01N 55/00; A01N 25/34; A41D 13/11

(52) U.S. Cl. CPC A01N 55/00; A61F 9/029; A41D 13/1192; A01N 25/34

(57) ABSTRACT

Personal Protective Equipment (PPE) is treated with a solution containing a quaternary ammonium compound bonded to a silane. The solution imparts antimicrobial and antiviral qualities to the PPE. The method includes soaking the PPE in the solution and drying the PPE, which imparts a long-lasting coating of the compound to the PPE. The treatment is useful for treating face masks, gloves, gowns, face shields, head coverings, foot coverings, and any other objects that require antiviral treatment.

### CROSS-REFERENCE TO RELATED APPLICATIONS

This application claims priority under 35 USC 119(e) of U.S. Provisional Patent Application Ser. No. 63/018,827, filed on May 1, 2020, the disclosure of which is herein incorporated by reference.

### BACKGROUND OF THE INVENTION

The present invention relates generally to a method for improving personal protective equipment. More specifically, the present invention is a method and system for improving the personal protective equipment of healthcare workers and the general public using antimicrobial technology additives.

With the outbreak of the Covid-19 pandemic, the spread of airborne pathogens is of great concern because of its potential impact on public health. Traditionally, face masks act as barrier masks and work by filtering particles out of the air. Thus, airborne pathogens can be filtered out. N85 masks are particularly effective. With the emergence of resistant and mutated strains, the use of conventional face masks comes into question as to the viability of pathogens surviving on the face mask surface, and the possibility of re-aerosolization of settled particles on a mask. Thus, it is imperative to devise a safe and effective alternative means to provide long-lasting antimicrobial protection on the surface of a mask, as to place an impact causing deactivation of pathogens when in contact with a mask.

Traditionally, the EPA defines “antimicrobial” as an agent that destroys and/or inhibits the growth of micro-organisms and related pathogens. In 2006, the testing of the active ingredient 3-(trimethoxysilyl) propyldimethyloctadecyl ammonium chloride showed anti-viral activity. The use of antiviral agents on inanimate surfaces has long been a subject discussed amongst scientists.

Viruses are made up of a lipoprotein envelope consisting of a protein-like membrane containing RNA and DNA material. This chemical make-up makes viruses vulnerable to antiviral agents. By using anti-microbial formulas that are highly effective and durable, that safely bond to targeted substrates, one can create a new antimicrobial surface that provides residual protection. This static antimicrobial surface coating can reduce the risk of exposure from unwanted pathogens. Antimicrobial technology can modify surfaces, on a molecular level, rendering surfaces free of microbial contamination and cross-contamination. This anti-microbial protection is strong, durable, and invisible, effective against most bacterial pathogens, fungus, and viruses. Recently, microbial contamination and cross-contamination have become a national and public concern in the spread of infectious diseases. Once applied to the surface/substrate, the active ingredient kills the pathogens, forming a protective/invisible protectant on the surface of the substrate.

### SUMMARY OF THE INVENTION

It is therefore an object of the invention to provide personal protective equipment (PPE) that is treated with antimicrobial agents in order to protect the wearers as well as the general public from transmissible diseases.

This invention addresses the need to improve Personal Protective Equipment (PPE) using cutting edge technology to enhance and protect healthcare workers, first responders, and the general population/consumer market. PPE are devices/appliances designed to be worn or held by an individual for the protection against one or more health or safety issues. The different types of PPE include face shields, gloves, eye gear, gowns, head and shoe covers, and masks. All protect against the transmission of pathogens through direct/indirect contact and respiratory droplets. Traditionally, PPE products are created to be utilized for varying degrees of implied pathology. The degree of barrier protection changes with the type of materials used and the amount of specific layering within the product. Thus, particle size can be controlled entering the skin, nose, and mouth. The level of PPE in healthcare is based on critical needs. It is worn to protect both the healthcare worker (occupational safety & health) and the patient as a means of infection control and prevention, keeping both the healthcare provider and the patient safe from each other. The use of PPE is obvious when there is an increased risk of high-velocity blood/saliva transmission in protecting the eyes, nose, mouth, and body protection. Respiratory masks (N95) limit the particle size from entering the respiratory system. Gloves are also an important component and an essential factor in PPE use. It is most important to keep healthcare workers healthy to ensure the vitality of those seeking care. It is imperative that PPE products like masks and gloves be made available to both health workers and patients to protect against unwanted pathogens and cross-contamination. Occupational exposures from patients with bloodborne and airborne pathogens are on the rise and must be addressed in a more technological manner. Traditionally, the fabric/woven PPE products relied on barrier protection techniques, including additional layering construction.

It is the intent of this invention to improve existing PPE products, by incorporating anti-microbial agents, providing increased efficacy in controlling the spread of diseases and cross-contamination of these pathogens. Textiles, whether woven or not, natural or synthetic, are widely used in the medical industry and the public safety arena. With an increase in public awareness on the spread of pathogens, there has been a greater demand for textiles that are more protective. Traditionally, metallic salt solutions like copper, Zinc, and Silver have been found to have antibacterial action on treated fabrics.

The PPE products according to the invention will give additional protection to help prevent the spread of pathogens, inhibiting microorganisms, fungus, and viruses from spreading. According to the invention, the PPE products (fabric or non-fabric) are treated with a water-based solution of silicone quaternary ammonium salt, producing a durable/long-lasting micro-static coating on the textile surface, integrated onto the substrate during or after the manufacturing process. When applied to the PPE surface, the antimicrobial forms an invisible (one molecule deep) layer that provides durability via a process of ion exchange and rapid polymerization, resulting in antimicrobial protection. For example, the antimicrobial solution to the inside and outside of an N95 or surgical mask will increase the protective properties of the mask, protecting both the healthcare workers and the patient simultaneously. When the fabric is treated with a combination of a silane base and antimicrobial agent, the silane base enables the antimicrobial agent to bond securely to the substrate surface, forming a covalent bond via a series of hydrolysis reactions. This provides the long-lasting antimicrobial protection against a broad spectrum of single-cell micro-organisms. This added technology to PPE devices will protect healthcare workers, first responders, patients, and the general public from the pathogenic transmission of diseases and cross-contamination. When these micro bacteria and viruses hit the mask (inside and outside), the offending microbes are lysed and destroyed by the mode of action that is electromechanical in nature. Because the silane base enables the antimicrobial agent to attach to almost any substrate/surface, the same technology can be applied to gloves (or gowns head covers, shoe covers). The technology can be added to fabric gloves, latex, rubber, vinyl, polypropylene, and nitrile. Because the antimicrobial component attaches so easily to the surface of the gloves, this added protectant makes it perfect for preventing infectious contamination of pathogens directly and indirectly and helps prevent cross-contamination.

It is the purpose of this invention to make an improvement on an already existing idea. Personal protection equipment/devices such as medical masks, surgical masks, face masks, or gloves (fibers, latex, vinyl, nitrile, polyethylene, etc.) were designed to protect against unwanted airborne pathogens/fluid droplets from entering the respiratory system and in the case of gloves prevent the spread of pathogens directly or in directing from contact contamination of a surface. Previously, the mode of action for masks to reduce pathogens was via a filtration mechanism, acting as a physical barrier. (blocking particles from entering). By incorporating an antimicrobial (silicone quaternary ammonium salt) to a bonding component (silane base), the covalent bond that is formed on the surface of the mask (applied inside and outside) will provide an enhanced long lasting antimicrobial protection against a broad spectrum of single-cell organisms, not excluding viruses like Covid (SARS-CoV-2). This addition to personal protection devices (applied to masks and gloves of any material) will provide the added protection to keep health care workers, patients, and the general public from unwanted pathogenic diseases and cross-contamination. Thus, when the pathogens hit the PPE devices, the offending microbes will be destroyed by an electro-mechanical mode of action. Thus, the incidence of repeated contamination and cross-contamination will be substantially diminished. This process can be performed prior to the manufacturing procedure, during the manufacturing procedure, or after the manufacturing procedure. In a world where the risk of acquiring unwanted pathogens is on the rise, it is necessary to provide better protection not only to the healthcare workers but also to the General public, in both public and private industry. Treating PPE (masks, Gloves, gowns) will reduce the spread of diseases and help address cross-contamination of pathogens.

Some of the areas where PPE will be beneficial: 1) Health care 2) Food Industry including servers, restaurants, buffets 3) Public Transportation such as buses, trains, 4) sporting arenas, theaters, casinos 5) airline industry, travel and hospitality 6) service industries such as hair salons, barber shops, tattoo and massage parlors 7) bowling alleys.

### BRIEF DESCRIPTION OF THE DRAWINGS

The invention will be described with reference to the drawings. The drawings are to be considered as illustrations of individual embodiments and not as limits to the claims.

In the drawings, where similar reference numerals depict similar elements:

FIG. 1 is an illustration of a mask being utilized by the present invention;

FIG. 2 is an illustration of another mask being utilized by the present invention;

FIG. 3 is an illustration of a glove being utilized by the present invention; and

FIG. 4 shows a pouch for use in treating the PPE according to the invention; and

FIG. 5 shows a tablet of antimicrobial compound used for creating the solution for treating the PPE.

### DETAILED DESCRIPTION OF THE INVENTION

All illustrations of the drawings are for the purpose of describing selected versions of the present invention and are not intended to limit the scope of the present invention. Although the invention has been explained in relation to its preferred embodiment, it is to be understood that many other possible modifications and variations can be made without departing from the spirit and scope of the invention.

The QAC/Silane base provides long-acting protection against the spread of germs that can cause cross-contamination. The mechanism of action of QAC/Silane base results in cell membrane rupture, from the electrostatic interaction between the Cationic QAC and the negatively charged bacterial outer cell membrane. The preferred QAC/Silane is 3-(trimethoxysilyl) propyldimethyloctadecyl ammonium chloride, which has the formula:

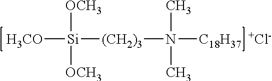

The QAC/Silane base is long-acting, lasting 30 to 90 days when applied to the surface of fabrics. The QAC/Silane can be mixed into a detergent solution containing water and/or other compounds. The standard application amount is 2 oz of QAC/Silane base detergent per 10 lbs of fabric

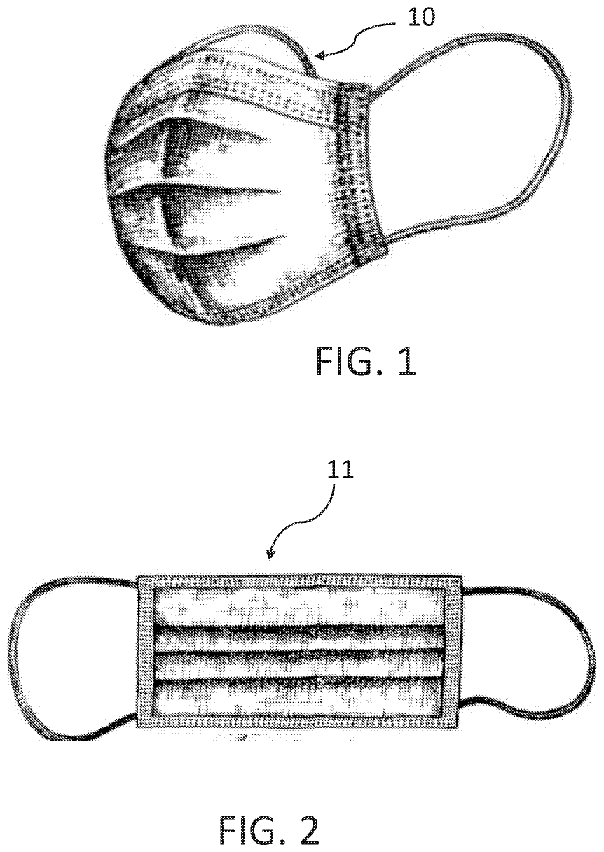

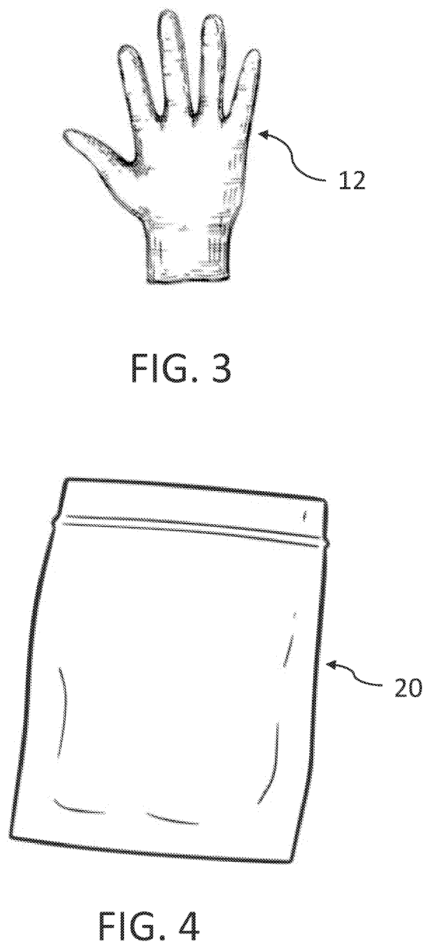

A finished face mask 10, 11 as shown in FIGS. 1 and 2 or glove 12 as shown in FIG. 3 can be treated with this solution. As shown in FIG. 4, one face mask 10 would require 5-10 drops of QAC/Silane base detergent placed in a translucent container 20, envelope, or pouch that is water-tight when closed. The container 20 is filled with water so that face mask is adequately covered, and the 5-10 drops are added. The container is shaken for 30-60 seconds. The formation of bubbles indicates activation by the compounds. The mask is then removed from the pouch, excess liquid is squeezed out and the mask is left to air dry or can be dried on low heat for 5-10 minutes.

Alternatively, the QAC/Silane Base can be applied directly to any fabric or thread prior to product manufacturing. The antimicrobial coating can be applied during the manufacturing process, by spraying or dipping or soaking the product in a QAC/Silane base solution. All surfaces are covered.

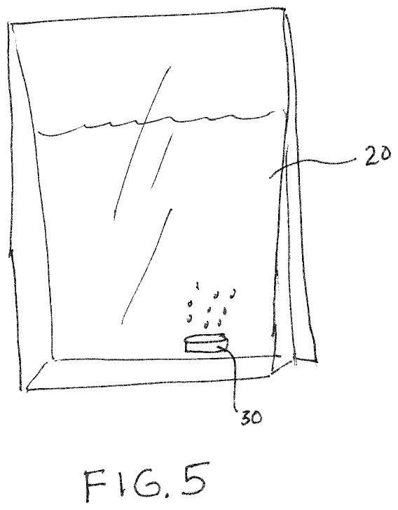

Alternatively, the QAC Silane can be delivered to the substrate using a water soluble paper that has been previously treated with an QAC/Silane base. These water soluble shaped Quaternary Ammonium/Silane discs 30 as shown in FIG. 5 will dissolve when placed in water. This method of delivering antimicrobial protection allows the user to further protect themselves by avoiding inhalation of the concentrated material. The disc 30 is dissolved in water in a watertight container 20 and then the article such as a face mask 10 is placed inside. The container is then shaken for 30 seconds to 1 minute. Once the disc is dissolved, antimicrobial protection is afforded. These discs 30 can also be placed in a washing machine.

What is claimed is:

1. A method of treating an article comprising soaking the article in a solution containing an antimicrobial compound in the form of a quaternary ammonium salt bonded to a silane base, and drying the article.

2. The method according to claim 1, wherein the antimicrobial compound is 3-(trimethoxysilyl) propyldimethyloctadecyl ammonium chloride.

3. The method according to claim 1, wherein the article is selected from the group consisting of a face mask, gloves, surgical gown, face shield, cap and protective eyewear.

4. The method according to claim 1, wherein the step of soaking takes place in a washing machine.

5. The method according to claim 1, wherein the step of soaking takes place in a water-tight pouch.

6. The method according to claim 1, wherein the step of drying comprises applying heated air to the article.

7. The method according to claim 1, wherein further comprising the step of forming the solution by dissolving a tablet containing the antimicrobial compound in water.

8. A method of producing an article comprising treating a base material of the article with a solution containing an antimicrobial compound in the form of a quaternary ammonium salt bonded to a silane base and then manufacturing the article from the treated base material.

9. The method according to claim 8, wherein the antimicrobial compound is 3-(trimethoxysilyl) propyldimethyloctadecyl ammonium chloride.

10. The method according to claim 8, wherein the step of manufacturing the article comprises manufacturing a face mask.

11. A face mask that is coated with or impregnated with a solution containing 3-(trimethoxysilyl) propyldimethyloctadecyl ammonium chloride.
